(19) United States
(12) Patent Application Publication
(10) Pub. No.: US 2019/0314043 A1
(43) Pub. Date: Oct. 17, 2019

(54) TOOL ASSEMBLY INCLUDING A CUTTING TOOL AND CUTTING GUIDE

(71) Applicant: Ronald Sekel, Kogarah (AU)

(72) Inventor: Ronald Sekel, Kogarah (AU)

(21) Appl. No.: 16/457,105

(22) Filed: Jun. 28, 2019

Related U.S. Application Data
(63) Continuation-in-part of application No. 14/894,409, filed on Nov. 27, 2015, now abandoned.

(30) Foreign Application Priority Data
May 28, 2013 (AU) 3013901899

(51) Int. Cl. A61B 17/17; A61B 17/16; A61F 2/46

(52) U.S. Cl. CPC A61B 17/1767; A61F 2002/4649; A61F 2/4644; A61B 17/1677

(57) ABSTRACT

A cutting assembly for cutting a tapered cut in a hard material includes a cutting tool and a cutting guide, the cutting tool including a cutting profile shaped to enable the creation of a tapered cut in said hard material, the cutting guide co-operating with the cutting tool and including a distal end which fits a cut profile formed by the cutting tool and a cutting guide opening which guides a secondary cutter introduced therein to complete the tapered cut.

### CROSS-REFERENCE TO RELATED APPLICATION

This is a continuation-in-part of U.S. patent application Ser. No. 14/894,409, a § 371 national stage of PCT/AU2014/000554 filed May 28, 2014, both herein incorporated by reference.

### BACKGROUND OF THE INVENTION

The present invention relates to tools and equipment for drilling holes more particularly to such tools capable of use, but not exclusively in orthopaedic surgery. The invention further relates to an assembly including a cutting tool and cooperating guide which allow a first cut using the cutting tool and a second cut using the cutting guide. Although the invention is particularly suited to bone cutting in at least orthopaedic surgery, it will be appreciated that the tools can be adapted for use in cutting hard materials other than bone. The invention will primarily be described with reference to its application in orthopaedic surgery and with particular reference to use in repair of the anterior cruciate ligament in the human knee. The invention further relates to an assembly which enables formation of a tapered cut using a first cutter and a co-operating guide which enables and provides alignment for a secondary cut by a second cutting tool. The assembly allows the creation of a cut profile in which the opening has a narrow region and a wide region resulting in a wedge shaped component.

### PRIOR ART

Cruciate ligaments occur in the knee of humans and other bipedal animals and in the neck, fingers, and foot. The cruciate ligaments of the knee are the anterior cruciate ligament (ACL) and the posterior cruciate ligament (PCL). These ligaments are two, strong, generally rounded, bands of varying cross sections along their length that extend from the head of the tibia to the intercondyloid notch of the femur. The ACL femoral attachment is lateral and the PCL is medial. Due to a twist or rotation along the length of the ligament the proximal and distal ends are disposed so they cross each other like the limbs of an X. In other words their respective anchorages are in a sense out of phase. This makes the cruciate ligament a complex structure with a complex anatomical geometry. The ACL and PCL remain distinct throughout and each has its own partial synovial sheath. Relative to the femur, the ACL keeps the tibia from slipping forward and the PCL keeps the tibia from slipping backward. It is a critical ligament for posterior/anterior knee stability. ACL injuries are among the most common knee injuries suffered by sports people. A ruptured ACL can occur by a movement as benign as a sudden change of direction or in more traumatic impact such as falling awkwardly in a tackle and abnormal knee extensions. Anatomical characteristics of a particular individual may predispose one individual to an ACL rupture more so than another individual engaged in the same knee extension, trauma, impact etc. After an ACL reconstruction patients have varying degrees of successful results in that some can return to high level sports whereas others must be content with retirement from sports. Prognosis post-surgery can depend upon the quality of the ligament repair and the method of repair or reconstruction used in surgical treatment. After surgery patients are concerned about the risk of repeating the injury particularly if attempting a return to high level sports, in which the knee is loaded and stressed. The incidence of Anterior Cruciate Ligament (ACL) tears requiring surgery is about 50 per 100,000 in the general population in Australia. Males are more likely than females to rupture an ACL as a result of sports injuries. Prognostic factors in determining outcomes of ACL repairs include age, gender, timing of surgeries and other concomitant injuries such as cartilage and meniscal injuries.

One of the difficulties faced by surgeons in ACL or PCL repair is the loss of natural cruciate ligament tissue when ruptured. Each individual has a cruciate of finite length. If ruptured, the tissue at opposing ends at the rupture becomes frayed. In a case where the elected surgical method requires the frayed ends to be sewn together the loss of tissue length can inhibit an optimal result and inevitably results in failure of the ligament to heal compromising knee stability and the development of instability. In cases where the cruciate ligament cannot be sewn back together grafts can be employed as a substitute for the ruptured cruciate ligament.

A knee reconstruction can involve repair of the cruciate ligament or use of grafts. A graft for example may be harvested from the patella tendon in the knee—a bone patellar tendon bone autograft,—or from the hamstring—autogenous hamstring tendons. Surgical outcomes can be dependent upon the type of graft used for the repair, age and gender of the patient, durability of the graft. In high level sports a patellar tendon graft may be the preferred selection but a hamstring graft can work just as well in male or female patients. In a graft repair the ends of the graft must be anchored to femoral and tibial bone to simulate as far as is anatomically possible the natural anchorage of the cruciate ligament to bone. Screws and rods are used to effect an anchorage of the new graft but a graft is not usually as strong as the natural anatomy of the cruciate ligament. As the cruciate ligament has a complex structure and a unique geometry a tendon harvested from another site although very often effective in a repair is not ideal.

An ideal repair method is to re-join if possible the anterior cruciate ligament in the knee if there is sufficient length of the ligament left after the rupture. It can be very difficult to re-join ruptured cruciate ends due to the loss of length on rupture indicated earlier. If possible, the ends are stitched together but they must be stretched to a tension beyond the cruciate ligament's normal rest tension. Post-operative physiotherapy is then used in an attempt to regain as much of the original knee function and range of movement existing before the ACL rupture.

The current gold standard treatment option for mid-substance ACL tear is reconstruction with patellar or hamstring tendon autografts. The operation generally yields good results in many patients, although it carries an approximately 3% revision rate at 5 years. The reported rates of patients returning to pre-injury activity levels range widely from 26% to 90%. That means 10-74% patients did not have excellent results after the operation, indicating that there is still room for improvements in regards to ACL repair procedures.

There are two intrinsic surgical difficulties encountered with the current standard surgical treatment of ACL rupture. Firstly, the human ACL is not a simple cylindrical structure but has a complex anatomy consisting of at least 2 rotary bundles which are difficult to replicate. Secondly, the ACL tibial and especially femoral attachment sites have an ellipsoid rather than a circular footprint which has not been successfully reproduced by the single, double or four stranded ACL graft repairs that are currently used.

Primary repair of the ACL trialled in the past encountered the problem of non-union and subsequent failure of the ligament. The reason that many intra articular tissues fail to heal has been attributed to the lack of blood supply and fibroblastic proliferation. However, histological studies of ruptured ACL revealed that the proliferation of fibroblasts and angiogenesis does occur in ruptured ACLs and therefore ACL should have the healing potential similar to other ligamentous tissues.

There are histological reasons which may explain why repaired ligaments fail.

1. The expression of actin-containing smooth muscle cells in the synovial tissue results in the retraction and the formation of a gap between the ACL ends which prevents the healing process;

2. The subsequent formation of synovial tissue over the discontinuous ruptured ACL ends has been postulated to further impede the ACL healing process. It is hypothesised that the overlay of the two ruptured ACL ends could potentially overcome the problem of the gap formation and non-union in primary ACL repair.

In mid-substance Achilles tendon ruptures, a primary repair yields excellent results as the frayed ends are sutured together in an overlaying manner. A significant outcome-modifying measure in the primary repair of an Achilles tendon is the ability to overlay the two ends by plantar-flexing the ankle joint. The same significant outcome-modifying measure has not been possible in the past with the primary repair of the ACL as the frayed ends could not be overlayed due to loss of length from rupture.

There is an ongoing need to constantly explore ways to improve the apparatus and equipment for Cruciate and other ligament repairs and to increase the success rate of surgical repair of ligaments. More generally there is an ongoing need to constantly improve tooling and cutting devices including, but not limited to, those used in orthopaedic surgery.

### SUMMARY OF THE INVENTION

The present invention provides a tool assembly for cutting an opening relatively hard materials such as bone and includes a cutter which forms an opening for a co -operating cutting guide having an opening which receives a second cutter which completes cutting of the opening. The invention also provides a cutting tool and guide assembly for creating holes including such holes required in bone surgery. The invention further provides an assembly including a cutting tool providing a first cut and co-operating guide which allows a second cut using a second cutting blade. Although the invention is particularly suited to bone cutting in at least orthopaedic surgery, it will be appreciated that the tools can be adapted for use in cutting hard materials other than bone. The invention will primarily be described with reference to use in repair of the anterior cruciate ligament in the human knee. The invention further relates to an assembly which enables formation of a tapered cut using a first cutter and a cooperating guide which enables and provides alignment for a secondary cut by a second cutting tool thereby creating a tapered wedge from the cut profiles. The assembly allows the creation of a cut profile in which the opening formed has a narrow region and a wide region resulting in a wedge shaped component.

With this in mind the present invention provides a surgical tool assembly including a tool which enables tapered cutting to allow advancement and retraction of a bone core with or without ligament or tendon attached during a surgical procedure for repair of a ligament or tendon. More particularly the invention provides a cutting tool and associated cutting guide, the tool capable of drilling out a bone core from surrounding bone to enable advancement and retraction of the core so that the distance between ends of a soft tissue structure such as a ligament can be adjusted to allow either abutment or overlap of ends during repair. The invention further provides an assembly which includes a tool which allows repair of a ruptured tendon without using a graft.

More particularly the invention provides a cutting tool and guide which enables the creation of a tapered bone core which can be freed from surrounding bone to enable advancement and retraction of the tapered core. The invention further relates to a surgical kit including a cutting tool and cutting guide tool which allows a user to fashion a tapered core of bone (with or without tendon or ligament attached) and which can move relative to surrounding bone for altering the distance between a soft tissue anchored to the core for repair of ruptured soft tissues such as ligaments and the like.

Outlined broadly below are embodiments and features of the invention to enable the invention to be better understood, and in order that the present contribution to and improvement over the current the art may be better appreciated. There are, of course, additional features of the invention that will be described hereinafter and which will form the subject matter of the claims.

In this respect, before explaining at least one embodiment of the invention in detail, it is to be understood that the invention is not limited in its application to the details of construction and to the arrangements of the components set forth in the following description or illustrated in the drawings. The invention is capable of other embodiments and of being practiced and carried out in various ways in various anatomical sites including in veterinary applications. Also, it is to be understood that the phraseology and terminology employed herein are for the purpose of description and should not be regarded as limiting. As such, those skilled in the art will appreciate that the conception, upon which this disclosure is based, may readily be utilized as a basis for the designing of other variations on the tool assembly, structures, methods and systems for carrying out the purposes of the present invention.

It is one an object of the present invention to provide a new and improved cutting tool which removes the practical disadvantages encountered in surgical repair of an ACL and other cruciate ligaments. It is a further object of the present invention to provide a new and improved tool assembly including the cutting tool and guide for the above purpose and which is of a durable and reliable construction. A further object of the invention is to provide tooling which enables an alternative to autographing a torn ACL, and enables end-to-end repair of the ACL itself. In the past, it has not been possible to primarily repair a torn ACL, as both ends are anchored to the bone, and the central body ligament shred cannot be over-lapped and repaired. By using a cutting tool and associated guide a tapered cut is created which allows advancing a tibia core of bone with the attached ACL stump, say 0.5-1.0 cm, it is then possible to arthroscopically (or open) repair the tear with overlap. By cutting a tapered cut (preferably trapezoidal or tapering cone shaped bone plug (with the ACL still attached)), it is possible to advance and lock the bone in its canal, fix it solidly, and repair the ligament while maintaining alignment of the natural complex orientation of the ACL bundles.

These together with other objects of the invention, along with the various features of novelty which characterize the invention, are pointed out with particularity in the claims annexed to and forming a part of this disclosure. For a better understanding of the invention, its operating advantages and the specific objects attained by its uses, reference should be had to the accompanying drawings and descriptive matter in which there is illustrated preferred embodiments of the invention.

In one broad form the present invention comprises:
an assembly for enabling a tapered cut in a material to be cut, the assembly comprising a cutting tool at least partially curved and including a first end which engages a powered activation tool a second end which includes a cutting edge profile, a guide which co -operates with the cutting tool and insertable into a first cut profile made by the cutting profile; the guide including an opening which receives and guides a second cutting implement which makes a secondary cut in the material, the first and second cuts creating a tapered element releasable from the surrounding material.

According to a preferred embodiment, the cutting tool comprises a generally arcuate body with a wide proximal portion tapering to a narrow distal portion. The body includes cutting edges extending from the proximal to the distal end with a cutting profile there along formed by an array of cutting teeth.

In another broad form the present invention comprises:
a cutting tool for cutting a tapered cut in a hard material, the tool comprising a tool body having a first end which includes a profile enabling engagement of the cutting tool with a power tool; a second working end including a curved and tapered region extending distally, the working end including at least one cutting edge formed by an array of cutting teeth; wherein the taper is formed by an incline in the cutting edge.

In another broad form the present invention comprises:
an assembly for enabling a tapered cut in bone, the assembly comprising a cutting tool at least partially curved having a first end which engages a powered activation tool and a second tapered end which includes a cutting edge profile, a guide which co -operates with the cutting tool and insertable into a first cut made by the cutting profile; the guide including an opening which receives and guides a second cutting implement which makes a secondary cut in the bone, the first and second cuts creating a tapered bone portion releasable from the surrounding bone.

According to a preferred embodiment the cutting tool creates a profiled cut which cut receives a cutting guide. The cutting guide comprises a curved tapered body and at one end an opening which receives extending therethrough a second cutting blade which completes the tapered cut formed by the cutting tool.

Preferably the power tool induces a reciprocating rotational cutting action in the cutting tool thereby creating an arcuate cut in the material. According to one embodiment the material cut by the cutting tool is bone.

Preferably the cutting tool at least partially curved having a first end which engages a powered activation tool and a second tapered end which includes a cutting edge profile. The cutting edge profile is formed by a continuous array of teeth extending from a proximal region on the tool to a distal end and then from the distal end to a second proximal region. The guide which co-operates with the cutting tool has a similar profile and is insertable into a first cut made by the cutting tool. The guide includes an opening which receives and guides a second cutting implement which makes a secondary cut in the bone, the first and second cuts creating a tapered bone portion releasable from the surrounding bone.

In another broad form the present invention comprises:
a cutting guide for a cutting assembly comprising a cutting tool and the cutting guide, the cutting tool enabling creation of a tapered cut in a hard material, the tool comprising a tool body having a first end which includes a profile enabling operable engagement of the cutting tool with a power tool; a second working end including an arcuate cutter having a tapered region extending distally, the working end including at least one cutting region formed by an array of cutting teeth; the cutting guide comprising a guide body having a proximal end and distal end, the distal end including an arcuate wall having a tapered region extending distally and shaped to enter and fit a first cut formed by said cutting tool, the proximal end of the guide having a guide opening which receives and guides a secondary cutter to complete the first cut.

According to one embodiment the hard material is bone.

In another broad form the present invention comprises:

an assembly for cutting bone during a ligament reconstruction procedure, the assembly comprising: a guide for insertion of a pilot wire;
a bone cutting tool operable by a power tool to effect a tapered cut in the bone;
a guide member insertable in the tapered cut made by the cutting tool; the guide member having an opening which receives a cutting blade which completes the tapered cut.

The cutting tool is adapted for cutting bone which retains part of a ligament. The invention described herein is adaptable for the repair/reattachment of a variety of ligaments when shortened on rupture.

The cutting tool and associated guide form a variety of prismic shapes including cylindrical or trapezoidal sections of bone isolated from a bone site. Preferably, the cut section of bone is one that has a ligament attached to one face so that the cut bone can advance in the direction of the ligament. According to a preferred embodiment the ligament is the cruciate ligament.

The present invention provides an alternative to the known prior art and the shortcomings identified. The foregoing and other objects and advantages will appear from the description to follow. In the description reference is made to the accompanying representations, which forms a part hereof, and in which is shown by way of illustration specific embodiments in which the invention may be practiced. These embodiments will be described in sufficient detail to enable those skilled in the art to practice the invention, and it is to be understood that other embodiments may be utilized and that structural changes may be made without departing from the scope of the invention. In the accompanying illustrations, like reference characters designate the same or similar parts throughout the several views. The following detailed description is, therefore, not to be taken in a limiting sense, and the scope of the present invention is best defined by the appended claims.

### BRIEF DESCRIPTION OF THE DRAWINGS

The invention will be better understood and objects other than those set forth above will become apparent when consideration is given to the following detailed description thereof. Such description makes reference to the annexed drawings wherein:

FIG. 1a shows an anterior elevation view of a knee joint.

FIG. 1b shows the knee joint of FIG. 1a enlarged to show the anterior cruciate ligament.

FIG. 2 shows the knee joint of FIG. 1b with a ruptured cruciate ligament and an assembly including a power drill operated coring tool for creating a displaceable core in tibial bone at the tibial end of the cruciate ligament.

FIG. 3 shows the knee joint of FIG. 1b with the core in tibial bone at the tibial end of the cruciate ligament advanced so the severed ends of the cruciate ligament are re engaged.

FIG. 4 shows a schematic elevation view of a core made in tibial bone in isolation from the knee joint.

FIG. 5 shows the core of FIG. 4 with ACL part axially advanced towards an opposing part of ACL to close a gap.

FIG. 6 shows a perspective view of the cutting tool according to a preferred embodiment.

FIG. 7 shows a side elevation view of the cutting tool of FIG. 6.

FIG. 8 shows a plan view of the cutting tool according to a preferred embodiment.

FIG. 9 shows an end view of the cutting tool.

FIG. 10 shows a perspective view of the cutting guide according to a preferred embodiment.

FIG. 11 shows a side elevation view of the cutting guide of FIG. 10.

FIG. 12 shows a plan view of the cutting guide according to a preferred embodiment.

FIG. 13 shows a first end view of the cutting guide.

FIG. 14 shows a second end view of the cutting guide.

FIG. 15 shows the proximal tibia with a guide wire inserted using an alignment jig into the centre of the tibia.

FIG. 16 shows the proximal tibia with the cutting tool inserted.

FIG. 17 shows the proximal tibia with the cutting guide and secondary cutter inserted.

FIG. 18 shows the proximal tibia with tapered bone core advanced after cutting.

FIG. 19 shows a schematic representation of a ligament anchored to bone and the proximal advance after cutting.

FIG. 20 shows one embodiment of the geometry, taper angle and dimensions of a cut bone section.

### DETAILED DESCRIPTION

The present invention will now be described in more detail according to a preferred embodiment but non limiting embodiment and with reference to the accompanying illustrations. The examples referred to herein are illustrative and are not to be regarded as limiting the scope of the invention. While various embodiments of the invention have been described herein, it will be appreciated that these are capable of modification, and therefore the disclosures herein are not to be construed as limiting of the precise details set forth, but to avail such changes and alterations as fall within the purview of the description. Although the assembly incorporating the cutting tool and cutting guide will be described with reference to its use in anterior cruciate ligament repair it will be appreciated that the assembly has other applications where tapered cuts and tapered cores are required.

One of the difficulties faced by surgeons in ACL or PCL repair is the loss of natural cruciate ligament tissue when ruptured. Each individual has a cruciate of finite length. If ruptured, the tissue at opposing ends of the rupture becomes frayed. In a case where surgical treatment requires the frayed ends to be sewn together the loss of tissue length can inhibit an optimal result and inevitably results in failure of the ligament to heal which compromises joint stability leaving the joint unstable. In cases where the cruciate ligament cannot be sewn back together grafts can be employed as a substitute for the ruptured cruciate ligament.

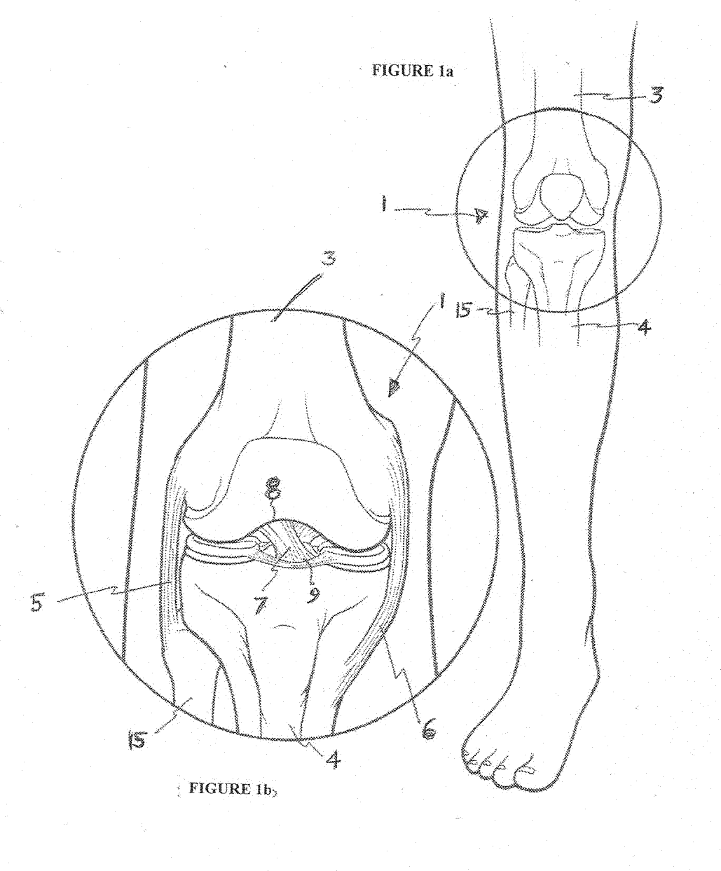

Referring to FIG. 1a and b there is shown an anterior view of a typical knee joint 1. Knee joint 1 is a schematic anatomical view of a right knee comprising a femur 3, tibia 4 and fibula 15. Joint 1 includes lateral collateral ligament 5 which connects femur 3 to tibia 4 and medial collateral ligament 6 which engages the femur 3 and tibia 4. The present invention will be described with reference to its application to the anterior cruciate ligament (ACL) 7 which is connected at one end 8 to the femur 3 and at the other end 9 to tibia 4.

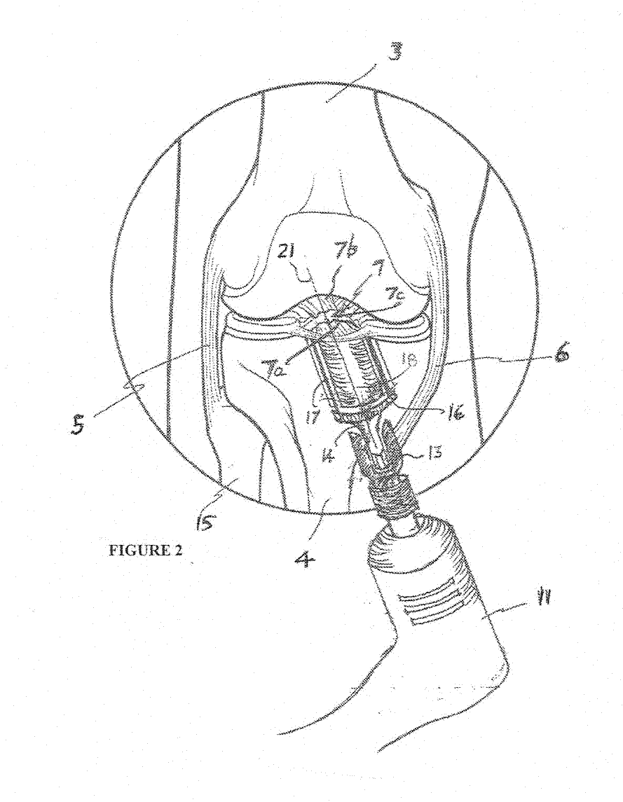

FIG. 2 shows the knee joint of FIG. 1b with corresponding numbering. ACL 7 is shown as ruptured into tibial part 7a and femoral part 7b leaving a gap 7c. Drill 11 is used in the repair of ACL 7a and 7b and comprises a drill 11 includes chuck 13. Chuck 13 retains a bit 14 comprising a base 16 which retains a coring tool 17 which allows a surgeon to drill a core 18 of tibial bone 4. Bit 14 and coring tool 17 are hollow to receive a pilot wire to initially establish an accurate path to the location of the ligament part 7a via the tibia. Core 18 when fully formed is free to advance and retract axially relative to tibia 4. Core 8 is preferably tapered so it can be arrested in its travel by surrounding bone. This allows the surgeon freedom to advance core 18 until part 7a of ACL 7 engages part 7b to close gap 7c. One of the problems faced by surgeons when attempting a knee reconstruction by re attaching the cruciate is the gap 7c between frayed ends 7a and 7b. This gap 7c represents a shortening of the ACL 7 which poses difficulty in connecting the ends 7a and 7b. The assembly according to the present invention described herein provides a method for drawing ends 7a and 7b of the ACL 7 closer together to thereby enable more ACL length for overlap and re attachment without having to stitch while the ACL would otherwise be in relatively high tension tending to pull the ends apart. FIG. 2 shows the core 18 prior to advancing tibial part 7a towards part 7b.

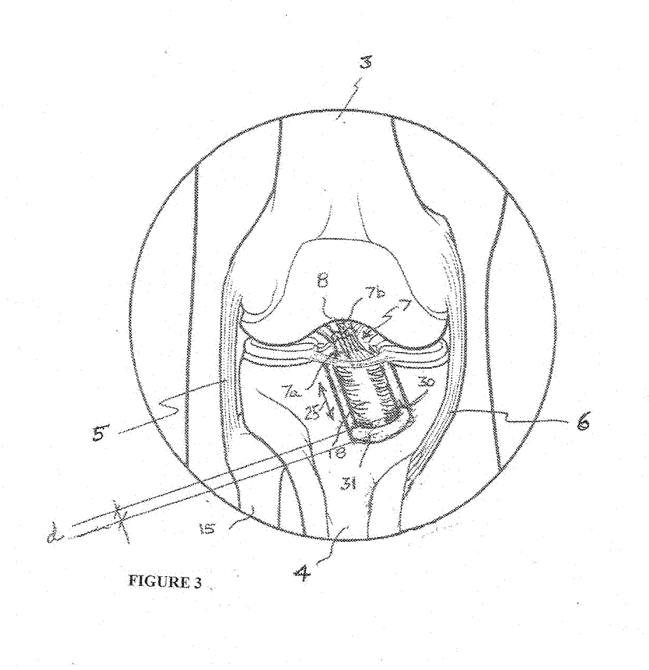

FIG. 3 shows with corresponding numbering the knee joint of FIG. 1b with the core 18 in tibial bone 4 at the tibial end of the cruciate ligament part 7a advanced so the severed ends 7a and 7b overlap to allow reattachment thereby closing gap 7c (FIG. 2). By using the appropriate instruments, a surgeon can achieve the advancement of conical core 18, overlay end 7a-to-end 7b followed by suturing and then re-tensioning of the repaired ACL 7 by retracting core 18 and reattaching it to tibial bone 4. One of the ways this may be achieved is by use of an anchorage rod (not shown) disposed transverse relative to the core 18. The new procedure allowed by the assembly 10 also eliminates the morbidity associated with the use of autografts in standard ACL reconstruction (ACLR) techniques (using bone-patellar-bone and hamstring harvesting techniques).

As shown in FIG. 3 core 18 advances axially in the direction of arrow 25 once coring tool 17 has created gap 30. Opening 31 allows a surgeon access to the core 18 so it can be advanced and retracted as required. Once ends 7 and 7b of ligament 7 are reattached, tension is re applied to ligament 7 to allows core 18 to be retracted back to as near as possible to its original location in the tibia 4. As ligament 7 is in that case under tension it will tend to pull core 18 towards the ligament due to the tension in the ligament. To resist this tendency, a transverse fixation or other suitable means for anchoring core 18 is adopted. Core 18 can advance at least as far as distance d which will preferably fall within the range of 2-10 mm.

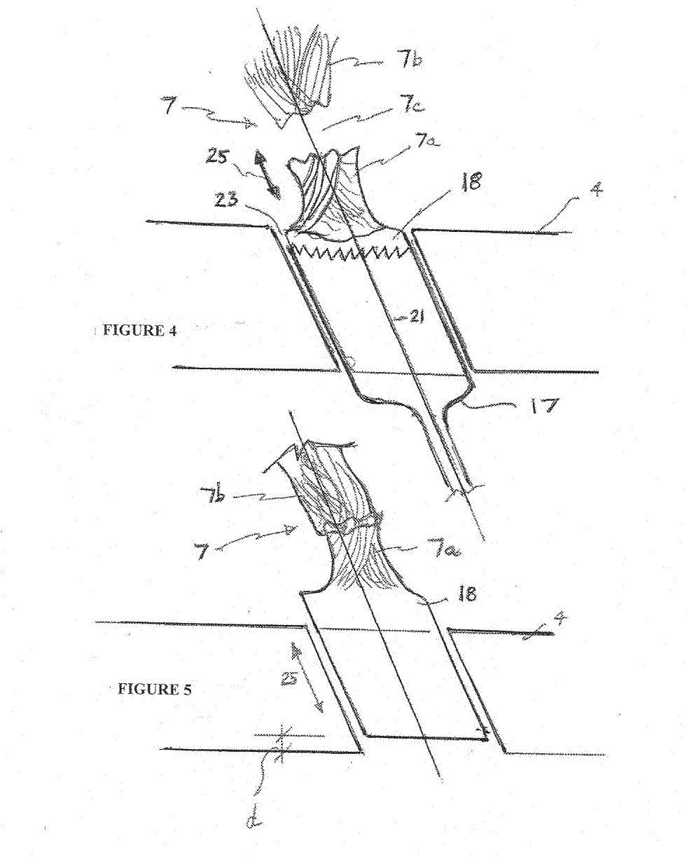

FIG. 4 shows a schematic elevation view of a core 18 made in tibial bone 4 in isolation from knee joint 1. The tool 17 is shown creating the core 18 by generating a circumferential gap 23 which isolates core 18 from tibia 4. Core 18 has extending therefrom a severed part of a cruciate ligament 7a which opposes corresponding end 7b of femoral end of ACL 7. As ACL 7 in FIG. 5 is shown ruptured a gap 7c is left requiring closure for repair. Core 18 in this view is shown as cylindrical but it will be appreciated that other shapes are contemplated such as but not limited to wedge shaped and trapezoidal

FIG. 5 shows the core 18 of FIG. 5 with ACL part 7a is axially advanced towards opposing part of ACL 7b to close gap 7c. Advancement of the core 18 in the direction of arrow 25 is enabled by the core drill bit 17 which separates core 18 from tibial bone. Core drill bit 17 may be a cylindrical crown bit or any attachment or bit capable of drilling a cylinder of bone. Core 18 is free to advance and retract as required by the surgeon over a range of 0-10 mm. The distance of axial movement of core 18 required is dictated by the size of gap 7c plus any overlap required to facilitate stitching/reconnection of the ACL parts 7a and 7b. FIGS. 1-5 show the concept of the ligament repair according to one embodiment including the movement of the bone core 18 when cut and released from surrounding bone. The bone core 18 shown is cylindrical but a tapered, trapezoidal or wedge shaped core is preferred. Set out below is a description of the invention with reference to a preferred cutting tool and cutting guide.

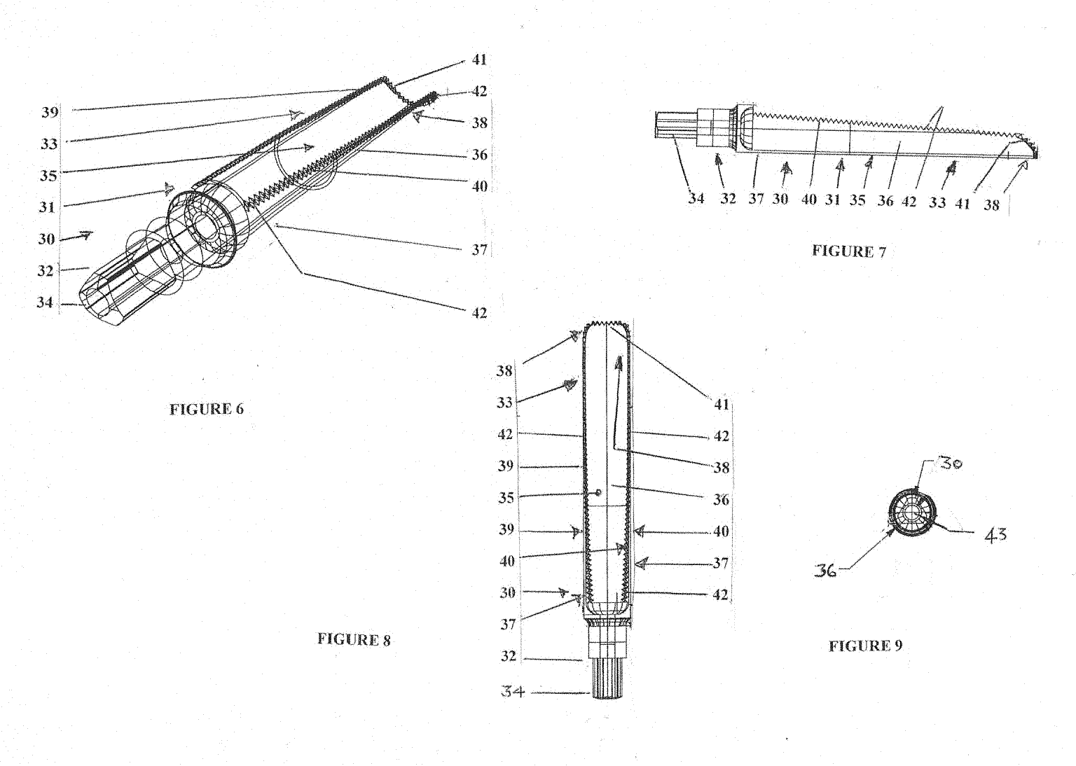

FIG. 6 shows a perspective view of the cutting tool 30 according to a preferred embodiment. Tool 30 comprises a body 31 having a proximal end 32 and distal end 33. Proximal end 32 includes a cannulated formation 34 which is insertable in and retained by a conventional driving tool such as a power drill. Distal end 33 comprises a cutter 35 having an abbreviated arcuate wall 36. Wall 36 has a proximal end 37 at which the circumference of the wall defines a circumferential arc within the range of 270-320 degrees and a distal end 38 at which the wall defines a circumferential arc less than that at the proximal end—preferably less than 180 degrees. This results in a tapering of the body 31 from the proximal end 37 to the distal end 38. Wall 36 terminates at lateral edges 39 and 40 and distally at edge 41. Each of edges 39, 40 and 41 have an array of cutting teeth 42. According to a preferred but non limiting embodiment, proximal end 37 comprises a wall arc greater than 180 degrees. Distal end 38 comprises a wall arc less than 180 degrees.

FIG. 7 shows with corresponding numbering a side elevation view of the cutting tool 30 of FIG. 7. From this view the tapering of the body 31 from the proximal end 37 to the distal end 38 is clearly shown. The taper of lateral edges 39 and 40 create a corresponding angled and tapered cut in bone.

FIG. 8 shows with corresponding numbering a plan view of the cutting tool 30. Leading edge 41 is transverse relative to the edges. 39 and 40 and provides a leading edge cut. FIG. 9 shows an end view of the cutting tool 30 showing cannulation 43.

FIG. 10 shows a perspective view of the cutting guide 50 according to a preferred embodiment. Guide 50 comprises a body 51 having a proximal end 52 and distal end 53. Distal end 53 includes a formation 34 which is insertable into a cut made by said cutting tool 40. Distal end 53 comprises a body having an abbreviated arcuate wall 55. Wall 55 has a proximal end 56 at which the circumference of the wall defines a circumferential arc within the range of 270-320 degrees and a distal end 57 at which the wall defines a circumferential arc less that that at the proximal end. This results in a tapering of the body 51 from the proximal end 56 to the distal end 57. Wall 55 terminates at lateral edges 58 and 59 and distally at leading edge 60. Wall 55 is insertable in cut made by cutting tool 40.

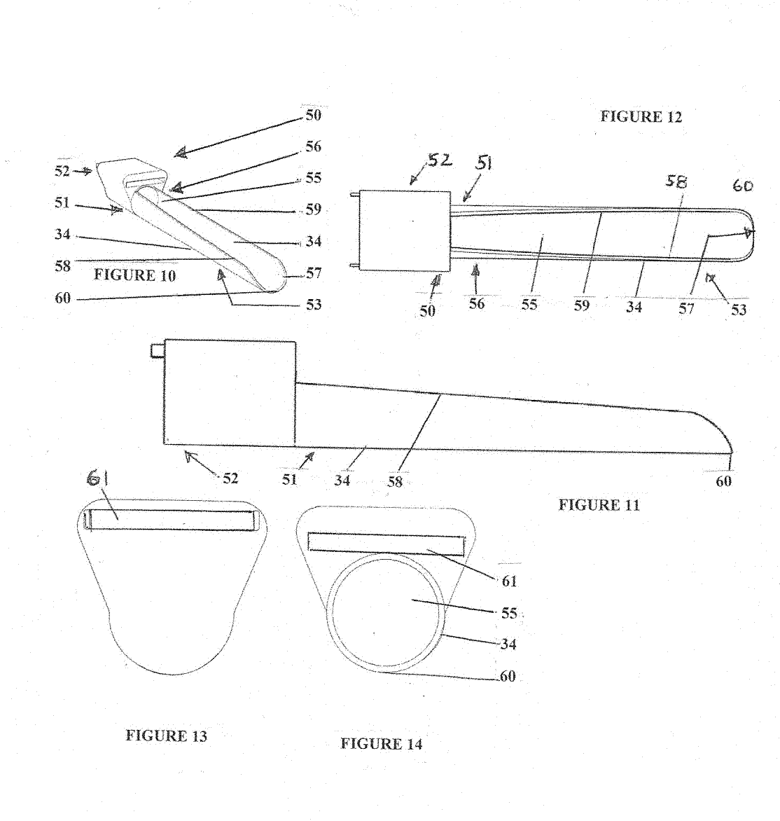

FIG. 11 shows with corresponding numbering a side elevation view of the cutting guide 50 of FIG. 7. From this view the tapering of the body 51 from the proximal end 56 to the distal end 57 is clearly shown.

FIG. 12 shows with corresponding numbering a plan view of the cutting guide 50. FIG. 13 shows a first end (proximal) view of the cutting guide 50. FIG. 14 shows a second (distal) end view of the cutting guide 50. Guide 50 includes a lateral slot 61 which receives a secondary cutting blade 62 (see FIG. 17).

The steps below highlight a preferred technique adopted using the cutting assembly. The surgeon arthroscopes the knee, then performs a mini open medial arthrotomy with a mini medial upper tibia incision. Using a standard Anterior Cruciate Ligament alignment jig, the length of the trapezoidal cone shaped piece of bone is measured from the length of wire internal exit point to the external tibia entry point. Using the ACL alignment jig (+/− computer guidance), the guide wire is drilled into the centre of the tibia attachment of the ACL. The ACL alignment jig, is removed leaving in the guide wire.

Using the guide wire the cannulated ˜300° rotating cutting tool 30 employs its cutting edges 39, 40 and 41 is advanced up to the inner tibial cortex, cutting an approximately 300° curved channel in the tibia. At this stage, the cutting is partially complete. A separate and secondary cut is required to complete release of the tapered bone core. Prior to the secondary cut a guide 50 is inserted into the curved cut made by cutting tool 30.

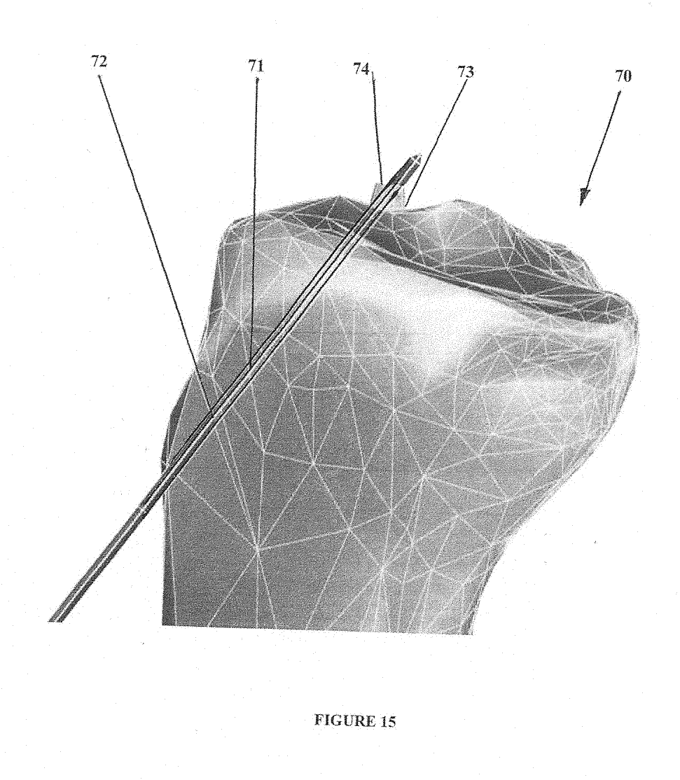

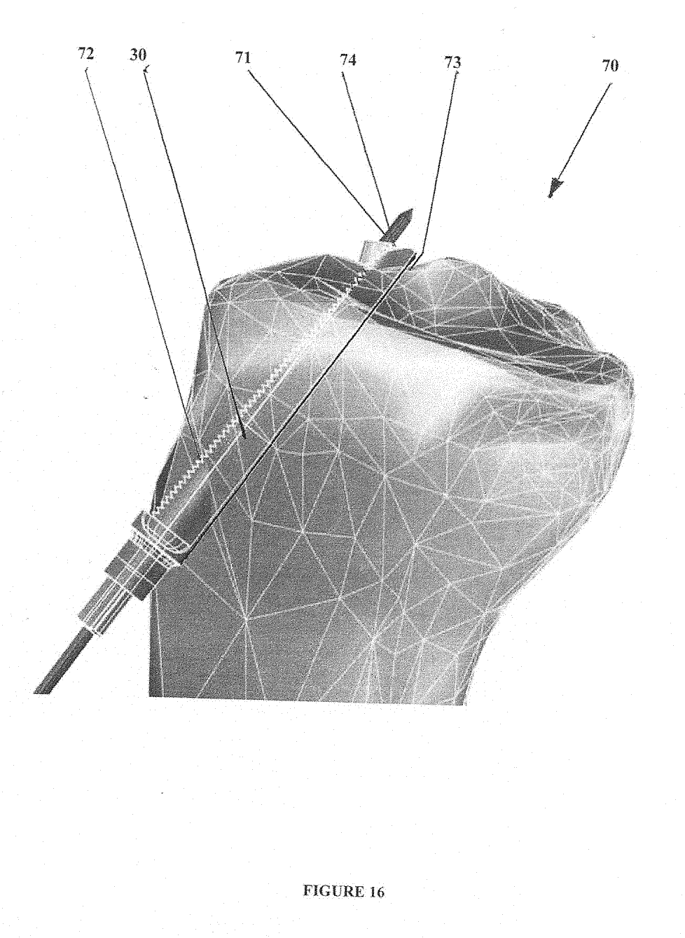

FIG. 15 shows the proximal tibia 70 with a guide wire 71 inserted using a known anterior cruciate ligament alignment jig into the centre of the tibia. Guide wire 71 enters at entry point 72 and exits at location 73 which is the distal anchor point for ligament 74. FIG. 16 shows the proximal tibia 70 with cannulated cutting tool 30 inserted. Purpose designed cannulated cutting tool 30 cuts a slightly greater than semicircular track in the bone. The cutting is enabled by a reciprocating and rotating power tool such as a drill. The cut formed in the bone is arcuate and tapers proximally to a narrow end. The narrow end is closest to the cruciate ligament.

FIG. 17 shows the proximal tibia with the cutting guide 50 and secondary cutter blade 62 inserted in lateral slot 61. With the cutting guide 50 inserted in the preformed cut and the secondary flat cutting blade 62 inserted via slot 61 into the cutting guide 50, cutting blade 62 cuts its way through the bone with its path assisted by guide 50. The tapering of the body 51 from the proximal end 56 to the distal end of the guide 57 is clearly shown. Body 51 is passed up the newly cut substantially semicircular track in bone. Body 51 has the same profile as the working end of cutting tool 30 and this enables body 51 to be inserted into the cut formed by cutting tool 30.

The cut formed by cutting tool 30 defines at its proximal end an arc greater than 180 degrees and at its distal end an arc less than 180 degrees. This naturally forms a tapered cut. Once guide 50 is in position, a known flat straight saw blade 62 cuts an oblique angled track accurately (−5 degree angle) into the bone. This secondary cut releases the cut bone core 80 (which is preferably in the form of a tapered cone) from its surrounding bone and enables the tapered bone component to advance in the direction of the anterior cruciate ligament. As a result of the secondary cut a flat region on the bone core prevents unwanted rotation of the cut bone core 80. It is desirable to prevent rotation of the freed bone segment 80 to avoid misalignment between the severed ends of the ACL. Bone core 80 is close to a cone shape and becomes wedged when it advances along the cut. The male cone shaped bone is advanced proximally after being advanced ˜0.5 cms. It then locks into the female similar shaped cone and is locked by an interference screw.

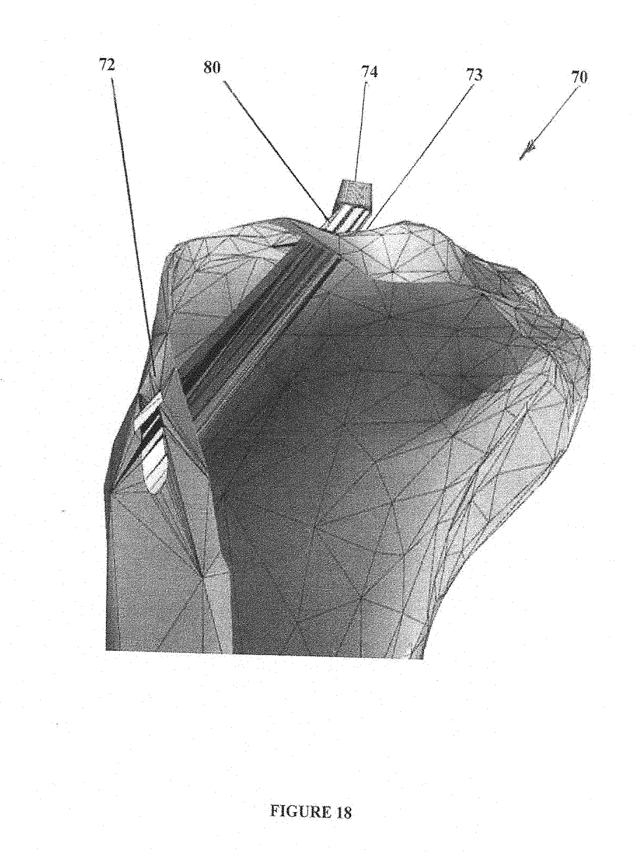

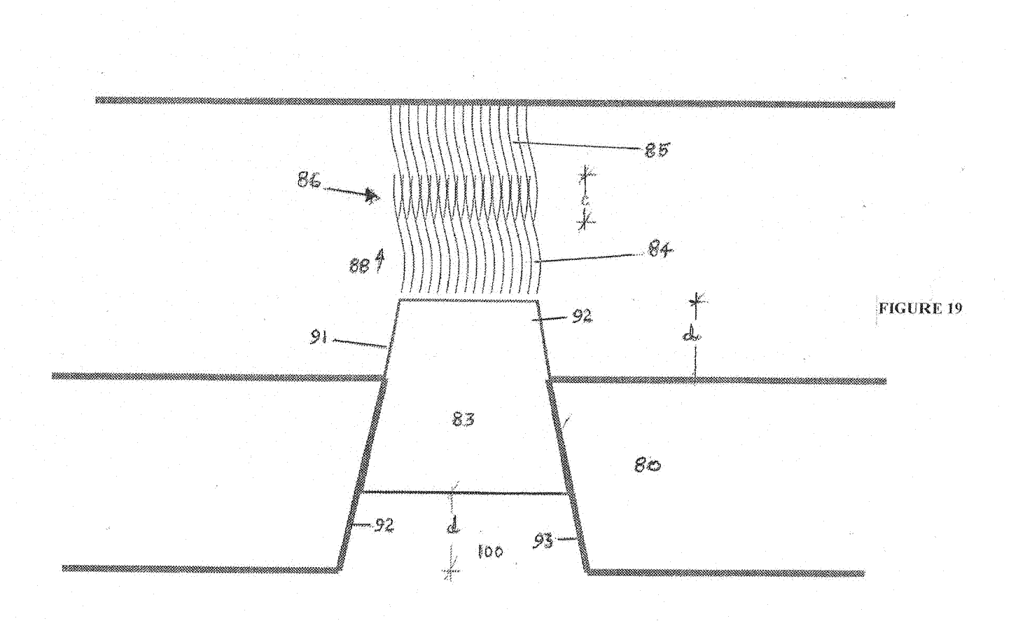

FIG. 18 shows the proximal tibia with tapered bone core 80 advanced after cutting. The ACL body or proximal tear is then repaired with a small amount of overlap, with the fibres in the correct original orientation.

With cutting tool 30 and guide 50 an angled track is cut to create the trapezoidal cone shaped bone (with the ACL attached) within the tibia. The trapezoid/cone of bone (with its attached ACL distal stump) is now free within its similarly shaped bone canal to be advanced in the direction of the ACL to allow an overlap which can then be repaired. The tapering bone+ACL stump is advanced about ˜½-1 cm to lock bone core 80 into the corresponding tapering tunnel in the tibia formed by the cut. The angle of the trapezoidal/cone shape has been calculated so that the bone+ACL stump does advance the required amount.

The two torn components of the ACL are now overlapped, ready for suturing. Optimally, bone anchors are implanted into the femur just adjacent to the proximal ACL footprint. The ACL is repaired with Bunnell type sutures, tied off at the base of the ACL footprint of the tibia. The sutures are optionally then passed down the sides of the trapezoidal tibial bone. The sutures are tied over an EndoButton after applying traction to tighten the ACL repair & the tibial bone segment.

FIG. 19 shows a schematic representation of a ligament anchored to bone and the proximal advance after cutting. Bone section 83 cut from tibial bone 80 and advanced to facilitate overlap of ruptured ends 84 and 85 of a cruciate ligament 86. In the embodiment shown bone section 83 is trapezoidal and has advanced a distance d from a pre cut position. Sides 90 and 91 of section 83 engage surfaces 92 and 93 respectively of tibia 80. Distance d will approximate the extent of overlap distance c between ruptured ligament parts 84 and 85. The cutting guide can allow various prismic shapes but the minimum width of the bone section will be dictated by the width of the footprint of the ruptured cruciate ligament. The smaller the angle of cut the greater the extent of travel of bone section 83. Likewise the larger the angle of cut the lower the distance of travel will be. The surgeon has flexibility to select from a kit of sizes of cutting guides the appropriate guide for a particular patent. Cruciate ligaments may vary in size from patient to patient requiring different cutting guide geometry. Also, in some cases only a small overlap of ruptured cruciate ligament will be required whereas in other a large overlap may be required depending upon how much cruciate is left after damage and the state of the ruptured ends.

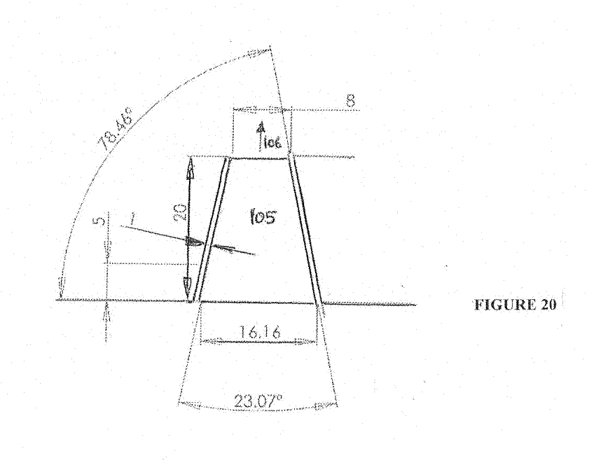

FIG. 20 shows one embodiment of the geometry, taper angle and dimensions of a cut bone section. The angle of cut according to this embodiment is 78.46 with a bone section maximum width 16.16 and a minimum width 8 mm. The gap between the tibial bone and the bone section cut is 1 mm. This geometry allows for an advancing distance of 5mm. Trapezoidal wedge 105 can advance in the direction of arrow 106.

One method of performing an ACL repair using the assembly and associated jig is described below. A minimally invasive mini medial arthrotomy incision is made approximately 3 cm in size. An ACL alignment jig is used to guide and drill a say 1.5 mm Kirschner wire into the centre of the proposed tibial bone core 80 exiting the centre of the distal ACL tibial attachment 7a. The bone core 80 containing the distal ACL segment 7a is cut in the tibia with the cutter tool 30 guided by the alignment of the pilot wire. The diameter of the tibial bone core cut by the crown saw would vary depending upon patient size but the size of core 80 would be in the region of 12, 14 or 16 mm depending on patient size.

Using a round punch, the tibial bone core is advanced up its tunnel for approximately 5 mm to allow overlap and end 7a-to-end 7b repair of the central body ACL rupture. A bone anchor is then inserted over each side of the proximal attachment of the ACL to the femur. Using a say Bunnell type suture, the two ends of the torn ACL 7a and 7b will be repaired via a medial arthrotomy. This may be performed using an arthroscopic procedure.

The bone core is then retrieved down its tunnel by either

a.) traction on the suture which is tied firmly over the tibia with an endobutton or similar apparatus; or
b.) pushing the tibial bone core down via access from the medial arthrotomy, leaving mild tension only at the repair site. The bone core is anchored onto the tibia with cross wires. The wounds are closed in the standard manner using sutures. The knee is immobilized in a hinge brace with the same postoperative program as that used for contemporary anterior cruciate repair (ACLR).

Using the core advancement technique the tibial bone core containing the distal ACL stump can be mobilized 3 to 5 mm proximally. An overlay repair of the two ruptured ACL ends 7a and 7b can be achieved The tibial bone tunnel containing the distal ACL stump can be fixed distally. The repaired ACL can be kept intact throughout the surgical procedure.

The following description sets out a series of preferred but non limiting steps which a surgeon may adopt when using the tooling described herein to create a bone prism whose free movement over a selected distance enables repair of a ruptured anterior cruciate ligament (ACL). A standard C Guide is used to set an alignment between a ruptured ACL and an axis which will indicate a path for a guide wire 71. The guide wire 71 traverses a path between the tibia and the ruptured ACL as described. This allows the surgeon to measure a distance a between an entry point in the tibia for the guide wire and the distal side of the ruptured ACL. A Computer guidance transmitter may be used to find an optimal angle of a block or wedge of bone to be drilled free of tibial bone. Distance a is a length between an entry point in the proximal tibia and a footprint centroid of ruptured ACL component anchored on the tibia. In a second step a guide wire 71 is inserted between the entry point and the centroid of the ACL component. The guide wire left in situ may have 0.5 mm laser markings or a depth gauge can be used to measure distance. The wire would have a known length. In a third step a slotted cutting block is urged against the tibial bone with its centre aligned with the path of the guide wire. Spikes are included on the plate spaced for centralizing the plate. For example four equally spaced spikes about 3 mm in length are provided. In a fourth step a cutting block is cut down on four sides of the block using a reciprocating saw using a 1 mm blade×5mm.

Once a cut has been formed in the bone the cutting device 30 is removed but the guide wire 71 is kept in situ. Secondary cutting guide 50 is inserted and a final flat cut is made with a known blade 62. The cut piece of bone which according to one embodiment is wedge shaped or a trapezoidal bone section advances proximally along the female passage preferably about 4 mm but within a range of 1-20 mm. When the bone section is advanced proximally, a transfixation wire is inserted transversely to lock the bone section from further movement relative to the tibia once ruptured ends of the cruciate ligament have been stitched together. Since the cut bone section is preferably wedge shaped—in that it has a wider distal end and a narrow proximal end. Since an inner wall of the tibia form which the bone section has been removed, is also tapered, movement of the bone section in the proximal direction will cause wedging of the section after it has advanced about 4 mm within the available range of movement. The cutting tool can be selected to release a bone section which achieves a desired limit of travel within the through passage.

The angle of an outer surface of the bone section will dictate the length of the travel within the passage formed. Selecting a cutting angle for the outer surface contour of the bone section will impact on the limit of travel. For example if a 2 cm block of bone cut from the tibia and having an apex of 8×6 mm, a taper angle of 27.5 would be required achieved by a 1 mm saw width and a 4mm advancement to interlock the bone section in the wedge shaped or trapezoidal shaped channel. Likewise, if a 3 cm block of bone cut from the tibia and having an apex of 8×6 mm, a taper angle of 27 would be required and achieved by a 1 mm saw width and a 4 mm advancement to interlock the bone section in the wedge shaped or trapezoidal shaped channel. If a 2.5 cm block of bone cut from the tibia and having an apex of 8×6 mm, a taper angle of 27 would be required and achieved by a 1 mm saw width and a 4 mm advancement to interlock the bone section in the wedge shaped or trapezoidal shaped channel. An acute taper angle would be about 27 degrees.

A surgeon would select an appropriate cutting guide based on the cutting angle required. Guides are provided at different angles and cut widths to control the extent of axial travel of the bone section cut. In the case of a cruciate ligament the footprint on the tibial bone can vary from patient to patient with typical cruciate base sizes in the range of 8-12 mm. Sizes outside this range are also contemplated. As well as selection of the angle of cutting guide the thickness of cutting blades also impact on the extent of axial advancement of the bone section cut. The thicker the cutting blade the longer the travel distance. 1mm wide cut may allow a 5-12 mm advance of the bone section. A preferred distance for advance of the bone section would be in the order of 5 mm at an angle of about 73-75 degrees. Also as bone has a certain elasticity this will also contribute to the overall extent of axial movement and can be allowed for in selection of cutting angles and thickness of cutting blades. Thus, it is proposed that a primary ACL repair using the research technique would bypass these intrinsic surgical difficulties as it does not require the surgeon to reproduce the ACL's complex multi strand spiral anatomy or ellipsoid attachment sites.

It will be appreciated by those skilled in the art that numerous variations and modifications may be made to the invention without departing from the overall spirit and scope of the invention broadly described herein.

1. A cutting assembly for cutting a tapered cut in a hard material, the assembly comprising a cutting tool and a cutting guide, the cutting tool including a cutting profile shaped to enable the creation of a tapered cut in said hard material, the cutting guide co-operating with said cutting tool and including a distal end which fits a cut profile formed by the cutting tool and a cutting guide opening which guides a secondary cutter introduced therein to complete the tapered cut.

2. The cutting assembly according to claim 1 wherein, the cutting tool comprises a tool body having a first end which includes a profile enabling operable engagement of the cutting tool with a power tool; a second working end including an arcuate cutter having a tapered region extending distally, the working end including at least one cutting region formed by an array of cutting teeth.

3. The cutting assembly according to claim 2 wherein the cutter further comprises an arcuate wall terminating in a wall edge.

4. The cutting assembly according to claim 3 wherein the wall at a proximal end has a greater arcuate extent than an arcuate extent at the distal end of the wall.

5. A cutting assembly according to claim 4 wherein the tapered region is formed by a gradual reduction in the arcuate extent of the wall about a longitudinal axis of the cutter from the proximal to the distal end of the wall.

6. The cutting assembly according to claim 5 wherein the cutting edge is continuous along the full extent of the wall edge.

7. The cutting assembly according to claim 6 wherein the cutting edge has cutting teeth extending along the full extent of the wall edge.

8. The cutting assembly according to claim 8 wherein cutting is enabled by reciprocating rotation of the working end.

9. The cutting assembly according to claim 8 wherein the cutting edge of the cutter comprises laterally spaced apart edges inclined from the distal end to the proximal end and each joined at a distal end by a transverse leading end cutting edge.

10. The cutting assembly according to claim 9 wherein the first end enabling engagement with a power tool comprises a member which engages and is retained by a drill chuck.

11. The cutting assembly according to claim 10 wherein the hard material is bone.

12. The cutting assembly according to claim 11 wherein the cutting guide comprises:
a guide body having a proximal end and distal end, the distal end including an arcuate wall having a tapered region extending distally and shaped to enter and fit a first cut formed by said cutting tool, the proximal end of the guide having a guide opening which receives and guides a secondary cutter to complete the first cut.

13. The cutting assembly according to claim 12 wherein the guide opening comprises a slot which receives a flat bladed cutting blade.

14. A cutting tool for a cutting assembly comprising the tool and a cutting guide, the cutting tool enabling creation of a tapered cut in a hard material, the tool comprising a tool body having a first end which includes a profile enabling operable engagement of the cutting tool with a power tool; a second working end including an arcuate cutter having a tapered region extending distally, the working end including at least one cutting region formed by an array of cutting teeth.

15. A cutting guide for a cutting assembly comprising a cutting tool and the cutting guide, the cutting tool enabling creation of a tapered cut in a hard material, the tool comprising a tool body having a first end which includes a profile enabling operable engagement of the cutting tool with a power tool; a second working end including an arcuate cutter having a tapered region extending distally, the working end including at least one cutting region formed by an array of cutting teeth; the cutting guide comprising a guide body having a proximal end and distal end, the distal end including an arcuate wall having a tapered region extending distally and shaped to enter and fit a first cut formed by said cutting tool, the proximal end of the guide having a guide opening which receives and guides a secondary cutter to complete the first cut.

16. The cutting guide assembly according to claim 15 wherein the hard material is bone.

17. An assembly for enabling a tapered cut in bone, the assembly comprising a cutting tool having a first end which engages a powered activation tool and a second curved tapered end which includes a cutting edge profile capable of creating the tapered cut in bone; a guide which co-operates with the cutting tool and insertable into a first cut made by the cutting tool; the guide including an opening which receives and guides a second cutting implement which makes a secondary cut in the bone, the first and second cuts creating a tapered bone portion releasable from the surrounding bone.

18. The assembly according to claim 17 wherein the cutting guide comprises a curved tapered body and at one end an opening which receives extending therethrough a second cutting blade which completes the tapered cut formed by the cutting tool.

19. The assembly according to claim 18 wherein the powered activation tool causes the cutting tool to reciprocate rotationally during cutting.

20. The assembly according to claim 19 wherein the tapered cut in bone is free to move relative to the surrounding bone in a passage in said bone created by the cutting tool and cutter using the cutting guide.

21. The assembly according to claim 20 wherein the tapered cut in bone is trapezoidal.

22. An assembly for use in a ligament reconstruction procedure after rupture of the ligament, the assembly comprising:
a jig capable of measurement of a length of a tibial bone section measured from a distal entry point to the tibia to a proximal location at which the length of bone is capable of separation form the tibia;
a guide wire for insertion axially through the bone section such that an entry point in the bone section aligns with a ruptured anterior cruciate ligament;
a bone cutting guide which has a first end which in use engages a tibial bone surface to be cut and a body which allows the bone section after cutting by the bone cutting element to be wider at a distal end than at a proximal end.

23. The assembly according to claim 22 wherein the bone cutting tool and guide enable creation of a trapezoidal profile shape of a bone section which allows the bone section when cut to advance a predetermined distance proximally relative to surrounding bone.
